(19) United States
(12) Patent Application Publication
(10) Pub. No.: US 2012/0096568 A1
(43) Pub. Date: Apr. 19, 2012

(54) TRANSGENIC LSD1 ANIMAL MODEL FOR CANCER

(76) Inventors: Roland Schuele, Weisweil (DE); Thomas Guenther, Freiburg (DE); Eric Metzger, Neuf-Brisach (FR)

(21) Appl. No.: 13/376,212

(22) PCT Filed: Apr. 28, 2010
(86) PCT No.: PCT/EP10/55720

§ 371 Date: Jan. 6, 2012

(30) Foreign Application Priority Data
Jun. 5, 2009 (EP) 09161995.7

(51) Int. Cl. A61K 49/00; C12N 15/85; A01K 67/027

(52) U.S. Cl. 800/3; 800/10; 800/21

(57) ABSTRACT

The present invention relates to a non-human transgenic animal whose genome comprises a stably integrated transgenic nucleotide sequence encoding Lysine-specific Demethylase 1 (LSD1) operably linked to a promoter. The invention further concerns methods for generating the non-human animal and its use as a cancer model.

### FIELD OF THE INVENTION

The present invention relates to an animal model for cancer which can be used to identify substances useful in the treatment of tumors and other proliverative disorders. In particular, the present invention relates to a non-human transgenic animal whose genome comprises a stably integrated transgenic nucleotide sequence encoding Lysine-specific Demethylase 1 (LSD1) operably linked to a promoter.

### BACKGROUND OF THE INVENTION

### LSD1

The DNA of cells of higher organisms is complexed by histone proteins and organized in chromatin. For this reason the organization and the dynamic regulation of the chromatin structure plays an important role in the control of transcription. The agglomeration and removal, respectively, of modifications of the termini of core histones results in a selective transcriptional response. We are just beginning to understand that these modifications of the chromatin do not only control the development and the function of an organism, but are also of great importance regarding the formation and the progression of tumours (Kouzarides, 2007).

Modifications may be effected e.g. by methylating lysine residues (K). The methylation of histones has been considered enzymatically irreversible for decades (Kouzarides, 2007). The discovery of the function of “amine oxidase (flavin containing) domain 2” (LSD1), which was the first histone demethylation, has proven this dogma wrong just a few years ago. LSD1 is able to demethlyate mono- and dimethylated lysine 4 and 9 in histone 3 (HK4, or H3K9 respectively), which is associated to a promoter, in a flavin-dependent mechanism (Shi et al. 2004, Metzger et al. 2005, Lee et al. 2005).

LSD1 is expressed in numerous tissues and is necessary for the normal growth of the mouse embryo. The deletion of this gene through homologous recombination leads to an early embryonic lethality of homozygous deficient mice. This makes it more difficult to analyze the function of LSD1 under normal and pathological conditions.

### Prostate Carcinoma

About 40,000 prostate carcinomas are newly diagnosed every year in Germany. Thus, this is the most frequent and most malignant carcinoma among men. The male sexual hormone, testosterone, plays an important role in the formation and the proliferation of the prostate carcinoma. Testosterone, or rather its active metabolite dihydroxytestosterone, executes its function via the androgen receptor (AR), which functions as ligand-dependent transcriptional factor in the nucleus and controls the differentiation, or the proliferation respectively, of normal prostate cells as well as of prostate carcinoma cells by controlling the expression of target genes. Therefore, the therapy of prostate carcinoma especially aims at decreasing the production of the male sexual hormone in the patients to inhibit the uncontrolled cell growth in the prostate gland. However, prostate carcinoma cells are also able to proliferate in the absence of androgens after some time (hormone refractory growth). At this stage of the disease, it is no longer possible to cure the patient (Sharifi et al., 2005).

### LSD1 Controls the Proliferation of Prostate Carcinoma Cells

LSD associates with AR on the chromatized DNA in the prostate cells. Dependent on the androgen receptor, this complex leads to the removal of the androgen repressive H3K9 methyl-marker through LSD1. This results in the activation of target cells of the AR, which is linked with an increased proliferation of prostate cell carcinoma in the cell culture system (Metzger et al., 2005, Metzger et al., 2008).

In a normal prostate, LSD1 is expressed in the endothel. However, the amount of the LSD1 protein is significantly increased in prostate carcinoma and correlates directly proportional with the malignity of the tumour and thus can be used as a tumour marker. In this connection, the observation that the reduction of LSD1 protein or the inhibition of its enzymatic activity in the cell culture system leads to a reduced proliferation, is particularly significant. This effect is restricted to prostate carcinoma cell lines expressing AR and does not occur in AR-deficient cells, whereby the deceleration of the cell growth through targeted inhibition of LSD1 is not dependent from androgens, because AR cannot be activated through steroid hormones anymore due to a mutation in C4-2B cells (Metzger et al., 2005; Kahl et al., 2006; Metzger et al., 2008). Thus, LSD1 offers for the first time a possible basis for decelerating the growth of prostate carcinoma cells even in the hormone refractory phase. Chemicals which inhibit the activity of these proteins in the cell culture system have already been tested in vitro and in the cell culture system on their effectiveness.

The inventors' observation that also in e.g. lung carcinoma and in the cell culture lines which have been derived thereof an significantly higher amount of LSD1 protein is present relating to inconspicuous tissue, indicates a potential significance of LSD1 extending beyond the prostate carcinoma. Moreover, this fact indicates that LSD1 does not exclusively conduct the control over the gene expression through AR. This hypothesis is emphasized by the observation that LSD1 interacts e.g. with “transformation related protein 53” (p. 53; Huang et al., 2007) and “retinoblastoma 1” (RB; Chau et al., 2008). Both gene products are essentially involved in controlling the cell cycle.

### BRIEF DESCRIPTION OF THE DRAWINGS

FIG. 1 depicts the demethylation of H3K4 catalyzed by LSD1. In a first step a histone (H3) is bound as a substrate and the methylated side chain is oxidized by the prostethic FAD group. Oxygen (O₂) is thereby reduced to H₂O₂. The resulting imine intermediate product is hydrolyzed, yielding a demethylated histone and formaldehyde (according to Formeris et al., 2008).

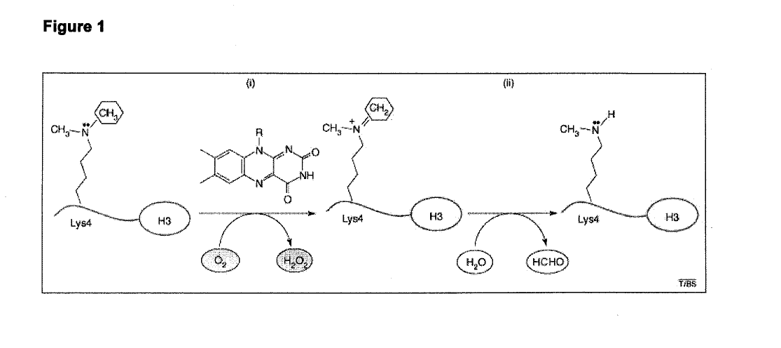

FIG. 2 depicts the detection of human FLAG-tagged LSD1 protein in various tissues from Rosa26-LSD1 transgenic mice. Immunoprecipitated protein extracts from various tissues were separated according to their size and transferred to a membrane. The Western blot was carried out using an antibody directed against the FLAG tag. In the first lane transiently transfected FLAG-LSD1 protein has been run as a control.

FIG. 3 depicts various tumors developed in adult Rosa26-LSD1 transgenic mice. Macroscopic (upper left panel) and microscopic (upper right panel) photographs of a bronchial adenocarcinoma are shown (arrows). Macroscopic image of a section through a liver lobe (bottom left panel) with hepathocellular carcinoma (arrow) is shown. The bottom right panel depicts a section through a peritoneal lipoma stained by haematoxylin and eosin.

FIG. 4 depicts a vector map of a recombinant construct (targeting vector) for generating transgenic mice by microinjection, see examples infra.

### SUMMARY OF THE INVENTION

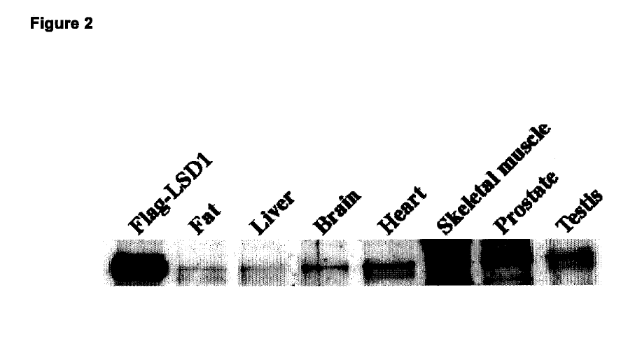

The inventors of the subject application found that LSD1 alone is sufficient for the generation of carcinoma. This was shown by generating transgenic mice carrying flag-tagged human LSD1 under the control of the Rosa26 promoter (Zambrowicz et al., 1997). These transgenic mice ubiquitously overexpress LSD1 protein (see FIG. 2).

Therefore, the transgenic mice of the present invention are a valuable tool for studying established and potential anti-cancer agents.

The present invention therefore relates to a non-human transgenic animal whose genome comprises a stably integrated transgenic nucleotide sequence encoding Lysine-specific Demethylase 1 (LSD1) operably linked to a promoter. The present invention is directed to a transgenic non-human animal, preferably a transgenic non-human mammal, whose genome comprises a DNA sequence encoding human LSD1 (=hLSD1) or an active fragment or variant thereof, which is operably linked to an expression control sequence, wherein the expression of the hLSD1 in the mammal is effective in stimulating the generation and/or growth of tumor cells or tissue. The transgenic mammal is a preferably a rodent, more preferably a mouse. The hLSD1 is preferably wild type hLSD1, and the DNA sequence may encode an active fragment or variant of hLSD1.

In the above transgenic mammal, the expression control sequence preferably comprises a constitutive promoter which may or may not be tissue specific; an inducible/repressible promoter or control element is also included. A preferred expression control sequence comprises a mouse Rosa26 promoter.

The transgenic mammal may be hemizygous for hLSD1, or more preferably, double hemizygous for hLSD1. It is preferably fertile.

In one embodiment of the above transgenic mammal, the polynucleotide was introduced into the animal, or an ancestor thereof, at an embryonic stage.

Also provided is a cell, isolated from the transgenic mammal, or a progeny cell of the isolated cell.

Also included is a method for testing an agent for its ability to inhibit the growth or metastasis of a human tumor, comprising exposing a transgenic mammal of the invention to the test agent, before or after development of a tumor in the mammal, and determining the effect of the test agent on tumor development and/or growth.

Also provided is a method for evaluating the effect of a test agent or treatment as a potential therapy for cancer, comprising administering a test agent or treatment to a transgenic mammal as above, and comparing the growth or metastasis of the tumor cell or tissue to a control transgenic animal.

Another embodiment provides a method for producing a transgenic mouse which is transgenic for LSD1, comprising incorporating into the genome of a mouse, at least one site, a polynucleotide encoding LSD1, or a biologically active fragment or variant thereof, which is operably linked to an expression control sequence, wherein the expression of the LSD1 in the mouse is effective to support the development and/or growth of tumor or cancer cells or tissue.

Also included is a method for preparing hLSD1 produced in a transgenic non-human mammal, comprising collecting an hLSD1-containing biological sample from the transgenic mammal as above, preferably a mouse, wherein the sample may be serum or plasma, and the hLSD1 in the sample is optionally further enriched or purified.

The invention further relates to the use of a non-human transgenic animal described herein as an animal model for cancer, e.g. for lung cancer, hepatocellular carcinoma and/or peritoneal lipoma.

The invention further relates to a recombinant nucleic acid construct for generating a nonhuman transgenic animal, said construct comprising a nucleotide sequence encoding LSD1 operably linked to a promoter, e.g the Rosa26 promoter. Said nucleotide sequence may be flanked by nucleotide sequences homologous to target sequences in the target animal. A preferred recombinant nucleic acid construct has the sequence as shown in SEQ ID NO:6.

### DETAILED DESCRIPTION OF THE INVENTION

The present invention relates to an animal model for evaluating growth, survival and/or metastasis of tumor cells or tissue. For example, the invention provides a transgenic non-human vertebrate animal, preferably a mammal, preferably a rodent, such as a mouse. In a most preferred embodiment, the genome of the animal comprises a polynucleotide which expresses the human growth factor, hLSD1.

One aspect of the invention non-human transgenic mammal (e.g., a rodent, preferably a mouse) whose genome comprises a DNA sequence encoding hLSD1, or encoding a biologically active fragment or variant thereof, which is operably linked to an expression control sequence, wherein overexpression of the hLSD1 leads to the development of tumors in the transgenic animal.

Suitable animals are available, or easily generated, using conventional methods, in a variety of genera, including rodents (e.g., rats), rabbits, guinea pigs, dogs, goats, sheep, cows, horses, pigs, llamas, camels or the like. Preferably, the non-human transgenic animal is a transgenic mouse.

The animal from which the progeny animal is descended is referred to as “progenitor animal.” “Progeny” of a progenitor mammal are any animals which are descended from the progenitor as a result of sexual reproduction or cloning of the progenitor, and which have inherited genetic material from the progenitor. In this context, cloning refers to production of genetically identical offspring from DNA or a cell(s) of the progenitor animal. As used herein, “development of an animal” from a cell or cells (embryonic cells, for example), or development of a cell or cells into an animal, refers to the developmental process that includes growth, division and differentiation of a fertilized egg or embryonic cells (and their progeny) to form an embryo, and birth and development of that embryonic animal into an adult animal.

An animal is “derived from” a transgenic ovum, sperm cell, embryo or other cell if the transgenic ovum, sperm cell, embryo or other cell contributes DNA to the animal's genomic DNA. For example, a transgenic embryo of the invention can develop into a transgenic animal of the invention. A transgenic ovum of the invention can be fertilized to create a transgenic embryo of the invention that develops into a transgenic animal of the invention. A transgenic sperm of the invention can be used to fertilize an ovum to create a transgenic embryo of the invention that develops into a transgenic animal of the invention. A transgenic cell of the invention can be used to clone a transgenic animal of the invention.

As used herein, a “transgenic non-human mammal” is a non-human mammal into which an exogenous recombinant construct has been introduced, or its progeny. Such a mammal may have developed from (a) embryonic cells into which the construct has been directly introduced or (b) progeny cells of (a). As used herein, an “exogenous construct” is a nucleic acid that is artificially introduced, or was originally artificially introduced, into an animal. The term “artificial introduction” excludes introduction of a construct into an animal through normal reproductive processes (such as by cross breeding). However, animals that have been produced by transfer of an exogenous construct through the breeding of a mammal comprising the construct (into whom the construct was originally “artificially introduced”) are considered to “comprise the exogenous construct.” Such animals are progeny of animals into which the exogenous construct has been introduced.

A non-human transgenic mammal of the invention is preferably one whose somatic and germ cells comprise at least one genomically integrated copy of a recombinant construct of the invention (a recombinant construct comprising a sequence encoding LSD, preferably hLSD1), or an active fragment or variant thereof, which sequence is operably linked to an expression control sequence. Alternatively, the disclosed transgene construct can also be assembled as an artificial chromosome, which does not integrate into the genome but which is maintained and inherited substantially stably in the animal. Artificial chromosomes of more than 200 kb can be used for this purpose.

The invention further provides a transgenic gamete, including a transgenic ovum or sperm cell, a transgenic embryo, and any other type of transgenic cell or cluster of cells, whether haploid, diploid, or of higher zygosity having at least one genomically integrated copy of a recombinant construct of the invention. The transgenic gamete, ovum, sperm cell, embryo, somatic cell or animal cell, may comprise two or more copies of the transgene. These are preferably tandemly arranged or may be inserted at noncontinguous sites in the haplotype (and genome).

As used herein, the term “embryo” includes a fertilized ovum or egg (i.e., a zygote) as well as later multicellular developmental stages of the organism. The recombinant construct is preferably integrated into the animal's somatic and germ cells, or is present in stable extrachromosomal form, such as an artificial chromosome, that is stable and heritable. The transgenic animal or cell preferably contains a multiplicity of genomically integrated copies of the construct. Preferably, multiple copies of the construct are integrated into the host's genome in a contiguous, head-to-tail orientation.

Also included herein are progeny of the transgenic animal that preferably comprise at least one genomically integrated copy of the construct, and transgenic animals derived from a transgenic ovum, sperm, embryo or other cell of the invention.

In some embodiments of the invention, the transgenic animal is sterile although, preferably, it is fertile. The present invention further includes a cell line derived from a transgenic embryo or other transgenic cell of the invention, which contains at least one copy of a recombinant construct of the invention. Methods of isolating such cells and propagating them are conventional.

While the mouse is preferred, the present invention includes other genera and species, such as other rodents (e.g., rats), rabbits, guinea pigs, dogs, goats, sheep, cows, pigs, llamas, camels, etc.

### Generation of Transgenic Animals

The transgenic non-human animals of the invention are produced by introducing transgenes into the germline of the non-human animal. Embryonal target cells at various developmental stages are used to introduce the transgenes of the invention. Different methods are used depending on the stage of development of the embryonal target cell(s). Such methods include, but are not limited to, microinjection of zygotes, viral integration, and transformation of embryonic stem cells as described below.

Microinjection of zygotes is the preferred method for incorporating transgenes into animal genomes. A zygote, which is a fertilized ovum that has not undergone pronuclei fusion or subsequent cell division, is the preferred target cell for microinjection of transgenic DNA sequences. The murine male pronucleus reaches a size of approximately 20 micrometers in diameter, a feature which allows for the reproducible injection of 1-2 picoliters of a solution containing transgenic DNA sequences. The use of a zygote for introduction of transgenes has the advantage that, in most cases, the injected transgenic DNA sequences will be incorporated into the host animal's genome before the first cell division. Brinster et al., Proc. Natl. Acad. Sci. USA 82:4438 (1985). As a consequence, all cells of the resultant transgenic animals (founder animals) stably carry an incorporated transgene at a particular genetic locus.

Viral integration can also be used to introduce the transgenes of the invention into an animal. The developing embryos are cultured in vitro to the blastocyte developmental stage. The blastomeres may be infected with appropriate retroviruses. Jaenich, Proc. Natl. Acad. Sci. USA 73:1260. Infection of the blastomeres is enhanced by enzymatic removal of the zona pellucida. Transgenes are introduced via viral vectors which are typically replication-defective but which remain competent for integration of viral-associated DNA sequences, including transgenic DNA sequences linked to such viral sequences, into the host animal's genome. Transfection is easily and efficiently obtained by culture of blastomeres on a monolayer of cells producing the transgene-containing viral vector. Alternatively, infection may be performed using cells at a later developmental stage, such as blastocoeles. In any event, most transgenic founder animals produced by viral integration will be mosaics for the transgenic allele; that is, the transgene is incorporated into only a subset of all the cells that form the transgenic founder animals. Moreover, multiple viral integration events may occur in a single founder animal, generating multiple transgenic alleles which will segregate in future generations of offspring. Introduction of transgenes into germline cells by this method is possible but probably occurs at a low frequency. However, once a transgene has been introduced into germline cells by this method, offspring may be produced in which the transgenic allele is present in all of the animal's cells, i.e., in both somatic and germline cells. Embryonic stem (ES) cells can also serve as target cells for introduction of the transgenes of the invention into animals. ES cells are obtained from pre-implantation embryos that are cultured in vitro. Evans et al., Nature 292:154 (1981). ES cells that have been transformed with a transgene can be combined with an animal blastocyst, after which the ES cells colonize the embryo and contribute to the germline of the resulting animal. Once a transgene has been introduced into germline cells by this method, offspring may be produced in which the transgenic allele is present in all of the animal's cells, i.e., in both somatic and germline cells.

The transgenic nucleic acid of the invention may be stably integrated into germ line cells and transmitted to offspring of the transgenic animal as Mendelian loci. Other transgenic techniques result in mosaic transgenic animals, in which some cells carry the transgenes and other cells do not. In mosaic transgenic animals in which germ line cells do not carry the transgenes, transmission of the transgenes to offspring does not occur. Nevertheless, mosaic transgenic animals are capable of demonstrating phenotypes associated with the transgenes.

In practicing the invention, animals of the transgenic maintenance line are crossed with animals having a genetic background in which expression of the transgene results in symptoms of tumor formation. Offspring that have inherited the transgenic nucleic acids of the invention are distinguished from littermates that have not inherited transgenic nucleic acids by analysis of genetic material from the offspring for the presence of nucleic acid sequences derived from the transgenic nucleic acids of the invention. For example, biological fluids that contain polypeptides uniquely encoded by the transgenic nucleic acids of the invention may be immunoassayed for the presence of the polypeptides. A simpler and more reliable means of identifying transgenic offspring comprises obtaining a tissue sample from an extremity of an animal, such as, for example, a tail, and analyzing the sample for the presence of nucleic acid sequences corresponding to the DNA sequence of a unique portion or portions of the transgenic nucleic acids of the invention. The presence of such nucleic acid sequences may be determined by, e.g., hybridization (“Southern”) analysis with DNA sequences corresponding to unique portions of the transgene, analysis of the products of PCR reactions using DNA sequences in a sample as substrates, oligonucleotides derived from the transgene's DNA sequence, and the like.

The present invention is also directed to the creation of transgenic mice in whose tissue specific expression of the hLSD1 transgene is driven by a tissue specific promoter, as is discussed more extensively below.

### Targeting Vectors

As used herein, the term “polynucleotide” is interchangeable with “nucleic acid.” A polynucleotide of the present invention may be recombinant, natural, or synthetic or semi-synthetic, or any combination thereof. Polynucleotides of the invention may be RNA, PNA, LNA, or DNA, or combinations thereof. As used herein, the terms peptide, polypeptide and protein are also interchangeable.

A “recombinant construct” (also referred to herein as a “construct” for short) or a “transgene” of “transgenic nucleic acid” which is used to generate a transgenic animal of the invention is a polynucleotide which comprises a sequence encoding LSD1 (preferably hLSD1), or an active fragment or variant thereof, which is operably linked to an expression control sequence. The coding sequence comprises LSD1 exon sequences, although it may optionally include intron sequences which are either derived from an hLSD1 genomic DNA or DNA of an unrelated chromosomal gene.

The recombinant construct may comprise a sequence encoding mLSD1 or at least a biologically active fragment thereof. Preferably, the recombinant construct comprises a sequence encoding human LSD1 (hLSD1) or a biologically active fragment thereof. The amino acid sequences of hLSD1 and mLSD1 are shown in SEQ ID NO:3 and 5, respectively. The nucleotide sequence encoding mLSD1 is shown in SEQ ID NO:4. The nucleotide sequence encoding hLSD1 is shown in SEQ ID NO:1. The hLSD1 cDNA sequence including 5′- and 3′-untranslated regions is shown in SEQ ID NO:2.

The nucleotide sequence of an exemplary recombinant construct is shown in SEQ ID NO:6. The amino acid sequence encoded by this construct is shown in SEQ ID NO:7.

A construct of the invention may comprise an “active fragment” or an “active variant” of a sequences encoding LSD1, e.g., hLSD1. Such an active fragment or variant encodes a form of LSD1 that exhibits at least a measurable degree of at least one biological activity of LSD1. For example, a polypeptide encoded by an “active” fragment or variant has enzymatic activity, in particular. A skilled worker can readily test whether a polynucleotide of interest exhibits this desired function, by employing well-known assays, such as those described elsewhere herein.

An active fragment of the invention may be of any size that is compatible with, for example, the requirement that it encode a polypeptide that can stimulate the growth of tumor cells. For example, an LSD1-encoding sequence can be shortened by about 20, about 40, or about 60 nucleotides, etc., provided that the encoded polypeptide retains the biological activity.

An active variant of the invention includes, for example, polynucleotides comprising a sequence that exhibit a sequence identity to DNA encoding wild type hLSD1, e.g., SEQ ID NO:1, of at least about 70%, preferably at least about 80%, more preferably at least about 90% or 95%, or 98%, provided that the polynucleotide encodes a polypeptide with the desired activity. In accordance with the present invention, a sequence being evaluated (the “Compared Sequence”) has a certain “percent identity with,” or is a certain “percent identical to” a claimed or described sequence (the “Reference Sequence”) after alignment of the two sequences. The “Percent Identity” is determined according to the following formula:

Percent Identity=100[1−(C/R)]

In this formula, C is the number of differences between the Reference Sequence and the Compared Sequence over the length of alignment between the two sequences wherein (i) each base in the Reference Sequence that does not have a corresponding aligned base in the Compared Sequence, and (ii) each gap in the Reference Sequence, and (iii) each aligned base in the Reference Sequence that is different from an aligned base in the Compared Sequence constitutes a difference. R is the number of bases of the Reference Sequence over the length of the alignment with the Compared Sequence with any gap created in the Reference Sequence also being counted as a base.

If an alignment exists between the Compared Sequence and the Reference Sequence for which the Percent Identity (calculated as above) is about equal to, or greater than, a specified minimum, the Compared Sequence has that specified minimum Percent Identity even if alignments may exist elsewhere in the sequence that show a lower Percent Identity than that specified.

In a preferred embodiment, the length of aligned sequence for comparison purposes is at least 30%, preferably at least 40%, more preferably at least 50%, even more preferably at least 60%, and even more preferably at least 70%, 80%, or 90% of the length of the Reference Sequence.

The comparison of sequences and determination of percent identity (and percent similarity) between two amino acid sequences can be accomplished using any suitable program, e.g. the program “BLAST 2 SEQUENCES (blastp)” (Tatusova et al. (1999) FEMS Microbiol. Lett. 174, 247-250) with the following parameters: Matrix BLOSUM62; Open gap 11 and extension gap 1 penalties; gap x_dropoff50; expect 10.0 word size 3; Filter: none. According to the present invention, the sequence comparison covers at least 40 amino acids, preferably at least 80 amino acids, more preferably at least 100 amino acids, and most preferably at least 120 amino acids.

The degree of identity between two polynucleotide sequences can be determined by using the program “BLAST 2 SEQUENCES (blastn)” (Tatusova et al. (1999) FEMS Microbiol. Lett. 174, 247-250) with the following parameters: reward for a match 1; penalty for a mismatch −2; open gap 5 and extension gap 2 penalties; gap x_dropoff 50; expect 10.0; word size 11; filter; none. According to the present invention the sequence comparison covers at least 50 nucleotides, preferably at least 100 nucleotides, more preferably at least 200 nucleotides; most preferably at least 300 nucleotides.

An active variant of the invention may take any of a variety of forms, including, e.g., a naturally or non-naturally occurring polymorphisms, including single nucleotide polymorphisms (SNPs), allelic variants, and mutants. The variant may comprise one or more additions, insertions, deletions, substitutions, transitions, transversions, inversions, or chromosomal translocations or the like; the variant may result from an alternative splicing event. Any combination of the foregoing is also intended. Other types of active variants will be evident to a person skilled in the art. For example, the nucleotides of a polynucleotide can be joined by known linkages, e.g., ester, sulfamate, sulfamide, phosphorothioate, phosphoramidate, methylphosphonate, carbamate, etc., depending on the desired purpose, such as improved in vivo stability, etc. See, e.g., U.S. Pat. No. 5,378,825. Any desired nucleotide or nucleotide analog such as 6-mercaptoguanine, 8-oxoguanine, etc. can be incorporated.

Active variants or fragments of the invention also includes polynucleotides which encode LSD1 polypeptides that differ from wild type hLSD1, yet retain at least one of the hLSD1 functions noted above. For example, the polypeptide may comprise a sequence that differs from the wild type hLSD1 sequence by one or more conservative amino acid substitutions, or that is at least about 70% identical, preferably at least about 80%, 90%, 95% or 98% identical, to the wild type sequence. The wild type sequence of hLSD1 is encoded by the cDNA having the sequence SEQ ID NO:1.

In the present recombinant construct, a hLSD1 coding sequence, or active fragment or variant thereof, is operably linked to an “expression control sequence”, which term means a polynucleotide sequence that regulates expression of a polypeptide from the coding sequence to which it is functionally (“operably”) linked. Expression can be regulated at the level of transcription or translation. Thus, an expression control sequence may include transcriptional elements and translational elements. Such elements include promoters, domains within promoters, upstream elements, enhancers, elements that confer tissue- or cell-specificity, response elements, ribosome binding sequences, transcriptional terminators, etc. An expression control sequence is operably linked to a nucleotide coding sequence when it is positioned in such a manner to drive or control expression of the coding sequence. For example, a promoter operably linked 5′ to a coding sequence drives expression of the coding sequence. One expression control sequence may be linked to another expression control sequence. For example, a tissue-specific expression control sequence may be linked to a basal promoter element.

Any of a variety of expression control sequences can be used in constructs of the invention. In preferred embodiments, the expression control sequence comprises a constitutive promoter, which is expressed in a wide variety of cell types. Many such suitable expression control sequences are well-known in the art. Among the suitable strong constitutive promoters and/or enhancers are expression control sequences from DNA viruses (e.g., SV40, polyoma virus, adenoviruses, adeno-associated virus, pox viruses, CMV, HSV, etc.) or from retroviral LTRs. Tissue-specific promoters well-known in the art maybe be used to direct expression of hLSD1 to specific cell lineages.

While the experiments discussed in the Examples below were conducted using the mouse Rosa26 gene promoter, other Rosa26-related promoters capable of directing LSD1 gene expression can be used to yield similar results as will be evident to those of skill in the art.

An example is shorter Rosa26 5′-upstream sequences, which can nevertheless achieve the same degree of expression. Also useful are minor DNA sequence variants of the Rosa26 promoter, such as point mutations, partial deletions or chemical modifications.

The Rosa26 promoter is known to be expressible in rats, rabbits and humans, and may be expressed in any other mammalian species, a fact which may be determined by routine testing. In addition, sequences that are similar to the 5′ flanking sequence of the mouse Rosa26 gene, including, but not limited to, promoters of Rosa26 homologues of other species (such as human, cattle, sheep, goat, rabbit and rat), can also be used. The Rosa26 gene is sufficiently conserved among different mammalian species that similar results with other Rosa26 promoters are expected.

For tissue-specific expression of the transgene in the transgenic animal, the coding sequence must be operably linked to an expression control sequence that drives expression specifically in that tissue. Suitable tissue-specific expression control sequences include the following: MMTV-LTR (for mammary-specific expression), etc.

### Inducible/Repressible Expression Control Systems

An inducible promoter is one which, in response to the presence of an inducer, is activated.

Hence, a coding sequence driven by an inducible promoter can be turned on or off by providing or withdrawing the inducer. A promoter may be homologous, derived from the same species as the coding sequence. Preferably, the promoter is heterologous, that is, derived from another species, or even from a virus. hLSD1 constructs in accordance with the present invention may be operably linked to an inducible or repressible control elements. An repressible system, described by Gossen, M. et al., Proc Natl Acad Sci USA 89:5547-51 (1992), is based on the use of control elements of the tetracycline-resistance operon encoded in Tn10 of E. coli. The tet repressor is fused with the activating domain of Herpes simplex virus VP16 to generate a tetracycline-controlled transactivator. Such a transactivator is used to stimulate transcription from a promoter sequence, such as the CMV promoter IE.

A gene controlled by a promoter acting under the influence of the tetracycline-controlled transactivator can be constitutively expressed and turned off by using an effective concentration of tetracycline. Such a system can regulate a gene over about five orders of magnitude. The tetracycline-repressible system functions in vivo in mice, where tetracycline administration via the diet is used to keep the expression of the inducible gene off. Tetracycline analogs which cross the blood-brain barrier can be used if gene activity is desired in the brain.

Two steps of transfection may be used to produce the appropriate system. A first transfection is used to isolate clones expressing the transactivator. The best clones are identified by testing each in a transient transfection assay for the ability to express a marker gene, such as an estrogen-dependent luciferase. The second transfection involves the hLSD1 coding sequence under control of an inducible promoter into a transactivator-containing clone. One strategy involves first isolating a stable cell line expressing the inducible hLSD1 protein or peptide by cotransfection of both plasmids into appropriate target cells. After selection, for example with G418, clones showing estrogen-dependent expression of hLSD1 may be detected by an immunoassay or biological assay. To increase the rate of plasmid integration and to stabilize the integrated plasmids in the host genome, the plasmids are preferably linearized and cotransfected into cells in the presence of mammalian high molecular weight DNA as a carrier.

The relative advantages of a two vector system, as described above, over a single vector system involving a larger plasmid is that in a two vector system, multiple copies of the reporter plasmid (encoding the gene of interest) may be needed to obtain a detectable biological effect in a cell, while one or only a few copies of the transactivator-carrying plasmid may suffice.

According to the present invention, the hLSD1 DNA molecule is placed under the control of a promoter subject to regulation by a tetracycline-controlled transactivator. Such a construct (in a single vector or preferably two vector form) is delivered into target cells, whether embryonic, adult normal or tumor, either in vitro or in vivo. To express the hLSD1, tetracycline is withheld so that the hLSD1 DNA is expressed. To prevent the action of the hLSD1, for example, locally, tetracycline or an active congener of tetracycline is administered locally to the cells transfected with the constructs. Effective systemic doses (oral or parenteral) of tetracycline are in the range of about 0.1 mg to 1 g per day. In a preferred embodiment, the transactivator is maintained in the “on” position by withholding tetracycline.

An estrogen-inducible system described by Braselmann, S. et al. Proc Natl Acad Sci USA (1993) 90:1657-61, is based on the fact that most mammalian cells neither express any Gal4-like activity nor endogenous estrogen receptor (ER), thus rendering estrogen an inert signal for them. The authors developed a selective induction system based on the estrogen-regulatable transcription factor Gal-ER. Gal-ER consists of the DNA-binding domain of the yeast Gal4 protein fused to the hormone-binding domain of the human ER and hence exclusively regulates a transfected coding sequence under the control of a Gal4-responsive promoter in mammalian cells. This system includes a synthetic Gal4-responsive promoter which consists of four Gal4-binding sites, an inverted CCAAT element, a TATA box, and the adenovirus major late initiation region. This promoter shows extremely low basal activity in the absence of, and high inducibility in the presence of, ligand-activated Gal-ER. The transcription factor Gal-ER is rendered more potent and less susceptible to cell type-specific variation by fusing the strong activating domain of the herpesvirus protein VP16 onto its C-terminus. In response to estrogen, e.g., 17-8 estradiol, Gal-ER-VP16 may induce the Gal4-responsive promoter at least 100-fold in transfected cells. Thus, the Gal-ER induction system is a powerful genetic switch for regulating heterologous genes. For induction of expression of the DNA molecules of the present invention in an estrogen inducible system in an animal, local or systemic treatment with estrogen would be required. An effective dose of an estrogen is a dose which would trigger the expression of an hLSD1-encoding nucleic acid of the present invention to produce hLSD1 and promote growth of hLSD1-expressing tumor cells. Such doses can be ascertained by one skilled in the art. Preferably, doses in the range of about 0.05 to 100 mg/kg of an estrogen are used in a single dose or in multiple doses over a period of about one week days to about 6 months, or even longer. Forms and preparations of estrogen and their usage in animals, particularly in humans, are well-known in the art. Estrogen analogues which are capable of specifically activating the exogenous transactivator while having fewer biological effects and side effects are preferred.

Ionizing radiation has been used to activate the transcription of exogenous genes, for example, encoding a cytotoxic protein TNF-I (Weichselbaum, R R et al., Int J Radiation Oncology Biol Phys 24:565-67 (1992)) This may be accomplished through the use of radiation-responsive elements distal to the transcription start site of such genes. See, for example, Hallahan, D et al., Proc Natl Acad Sci USA 88:2152-20 (1991); Datta, R et al., Proc Natl Acad Sci USA 89:10149-53 (1992); Weichselbaum et al., supra; Hallahan, D E et al. J Biol Chem 268:4903-07 (1993); Weichselbaum, R R et al., Intl J Radiation Oncology Bio. Phys 30:229-34 (1994); Hallahan, D E et al. Nature Med 1:786-91 (1995), which references are hereby incorporated by reference in their entirety. Thus, the present invention provides methods for the spatial and temporal control of gene expression with such radiation-inducible promoters to activate hLSD1. The hLSD1 coding sequence is placed in a vector under control of a radiation-inducible promoter.

Another generally applicable method is used in conjunction with gene therapy/gene delivery methods described below, for inducing activation of a gene of interest, in particular hLSD1. This method is disclosed in detail in PCT publications WO94/18317, WO95/02684 and WO95/05389; Spencer, D. M. et al., Science 262:1019-1024 (1993); Travis, Science 262:989 (1993); and Chem. & Eng. News, Nov. 15, 1993, pp. 55-57, which references are hereby incorporated by reference in their entirety. This approach uses intracellular protein homodimerization, heterodimerization and oligomerization in living cells into which the hLSD1 DNA has been transfected. Chimeric responder proteins are intracellularly expressed as fusion proteins with a specific receptor domain. Treatment of the cells with a cell-permeable multivalent ligand reagent which binds to the receptor domain leads to dimerization or oligomerization of the chimeric receptor. In analogy to other chimeric receptors (see e.g. Weiss, Cell (1993) 73, 209), the chimeric proteins are designed such that oligomerization triggers the desired subsequent events, e.g. the propagation of an intracellular signal via subsequent protein-protein interactions and thereby the activation of a specific subset of transcription factors. The initiation of transcription can be detected using a reporter gene assay. Intracellular crosslinking of chimeric proteins by synthetic ligands allows regulation of the synthesis of hLSD1 and, thereby, selective induction of tumor growth.

In a preferred embodiment, the expression control sequence (either a ubiquitously acting expression control sequence or a tissue-specific one) is expressed in a regulatable fashion, meaning that it is preferably a component of any of a number of well-known regulatable expression systems.

Methods of making recombinant constructs are conventional. Such methods, as well as many other molecular biological methods used in conjunction with the present invention, are discussed, e.g., in Sambrook, et al. (1989), Molecular Cloning, a Laboratory Manual, Cold Harbor Laboratory Press, Cold Spring Harbor, N.Y.; Ausubel et al. (1995). Current Protocols in Molecular Biology, N.Y., John Wiley & Sons; Davis et al. (1986), Basic Methods in Molecular Biology, Elsevier Sciences Publishing, Inc., New York; Hames et al. (1985), Nucleic Acid Hybridization, IL Press; Dracopoli et al. Current Protocols in Human Genetics, John Wiley & Sons, Inc.; and Coligan et al. Current Protocols in Protein Science, John Wiley & Sons, Inc. See, also, the Examples herein.

In a specific embodiment of this invention, the transgenic animal does not comprise additional transgenic sequences. This means that the transgenic LSD1 construct present in the transgenic animal is the only transgenic construct present in the animal of this invention. The transgenic animal preferably does not comprise further genetic germ line modifications other than that described hereinabove. The genetic background of the transgenic animal of the invention is ‘wild type’ except for the genetic modifications described hereinabove. This embodiment is preferred, as it allows studying the effects induced by LSD1 overexpression and of any methods interfering with the action of LSD1.

In another embodiment, the animal of the invention overexpresses one or more known oncogenes or protooncogens, or it comprises a knockout of one or more tumor suppressor genes. In yet another embodiment, the animal overexpresses one or more tumor suppressor genes.

### Method for Identifying Potential Therapeutic Agents

Another aspect of this invention is a method for identifying a compound which inhibits tumor growth, comprising (a) administering a test compound to a transgenic animal according to the present invention and (b) determining the effect of the test compound on the initiation, maintenance, or progression of cancer in said transgenic animal, thereby identifying a compound that inhibits tumor growth.

The method may comprise the steps (i) administering a test compound to a transgenic animal according to the present invention, (ii) administering the same test compound to a control animal, and (iii) determining the effect of the test compound on the transgenic animal as compared to said control animal. The control animal is preferably an animal of the same species as the transgenic animal. The control animal does not overexpress the polypeptide encoded by the transgenic nucleotide sequence of the test animal used in step (i) or it overexpresses it to a significantly lower degree (reduced by at least 25%, at least 50% or at least 75%), relative to the amount of polypeptide overexpressed in a given tissue of the transgenic test animal used in step (i). In a first embodiment, the control animal does not carry the transgenic nucleotide sequence present in the test animal of step (i). In another embodiment, the control animal is also carrying the same transgenic nucleotide sequence as the transgenic animal used in step (i) but there is no or only a weak overexpression. This can be achieved by the use of inducible transgene constructs, see supra.

The effect to be determined in step (iii) may be any change in any clinically or biologically relevant parameter including tumour mass, tumor size, presence of tumor markers, number of tumors, presence of metastases, survival rate and the like (relative to the control animal). The methods of analysis include but are not limited to determining the tumor mass, number of tumors, presence of metastases, tumor size, detecting tumor markers, histological methods and biochemical methods.

The test compound may be selected as a compound which inhibits tumor growth if there is a significant difference in at least one of the clinically or biologically relevant parameters tested, for example when the parameter (e.g. tumour mass, number of tumours, tumor size, tumor markers etc.) in the control animal is significantly reduced (e.g. by at least 10%, preferably by at least 25%, more preferably by at least 50%) relative to that of the transgenic test animal used in step (i).

The compounds used as test compounds may be inhibitors of LSD1.

LSD1 Inhibitors

The LSD1 inhibitor to be used in accordance with this invention is a compound capable of reducing the amount of LSD1 mRNA or LSD1 protein in a cell and/or inhibiting at least one function of the LSD1 gene or the LSD1 protein. These functions include (1) the ability of LSD1 to interact with androgen receptor, and (2) the catalytic activity of LSD1.

### 1. Compounds Capable of Inhibiting Expression of LSD1

The LSD1 inhibitor to be used in accordance with this invention may be a compound capable of inhibiting expression of the LSD1 gene in a cell. Various methods for inhibiting expression of genes in a cell are known to one of skill in the art. For example, expression of certain genes may be inhibited by using interfering RNA and/or antisense nucleic acids. It is preferred according to the present invention that the LSD1 inhibitor is selected from siRNA, shRNA, miRNA and antisense nucleic acids.

### a) Interfering RNA

Inhibitory double stranded nucleic acids (interfering nucleic acids, or siNAs) can also be used to inhibit gene expression, using conventional procedures. Preferably the inhibitory molecule is an short interfering RNA (siRNA) molecule. Typical methods to design, make and use interfering RNA molecules are described, e.g., in U.S. Pat. No. 6,506,559, U.S. Pat. No. 6,506,559; US Pat. publication 20030206887; and PCT publications WO99/07409, WO99/32619, WO 00/01846, WO 00/44914, WO00/44895, WO01/29058, WO01/36646, WO01/75164, WO01/92513, WO 01/29058, WO01/89304, WO01/90401, WO02/16620, and WO02/29858.

### b) Antisense Nucleic Acids

Antisense nucleic acids may be used to inhibit the expression of the LSD1 gene in a cell. Methods and techniques for designing and preparing antisense nucleic acids are known per se. The skilled person can provide suitable antisense nucleic acids on the basis of information derived from the nucleotide sequence of LSD1. Suitable techniques are described, e.g., in Asubel et al. ed., chapter 26 (1994-2008).

2. Compounds Capable of Inhibiting the Ability of LSD1 to Interact with Androgen Receptor.

In another embodiment the invention, the LSD1 inhibitor is capable of inhibiting the interaction of LSD1 protein with the androgen receptor. Such inhibitors include antibodies specifically binding to the LSD1 protein. The antibodies are preferably monoclonal antibodies. Techniques for generating monoclonal antibodies specifically binding to specific proteins are known to those of skill in the art (see, e.g., Harlow and Lane, Antibodies: A Laboratory Manual, Cold Spring Harbor 1988. Cold Spring Harbor Laboratory; or ULLMANN'S Biotechnology and Biochemical Engineering, pp 443-455, Wiley-VCH 2007). It is also possible to use antibodies directed against androgen receptor.

Another class of agents that can be screened for possible use as drugs are “small molecules,” also referred to herein as “compounds,” which are isolated from natural sources or made synthetically. In general, such molecules may be identified from large libraries of natural products or synthetic (or semi-synthetic) extracts or chemical libraries according to methods known in the art. Those skilled in the field of drug discovery and development will understand that the precise source of test extracts or compounds is not critical to the methods of the invention. Accordingly, virtually any number of chemical extracts or compounds can be used in the methods described herein. The types of extracts or compounds that may be tested include plant, fungal, prokaryotic or eukaryotic cell or organism-based extracts, fermentation broths, and synthetic compounds including modifications of existing compounds. Numerous methods are also available for generating random or directed synthesis (e.g., semi-synthesis or total synthesis) of any number of chemical compounds, including, but not limited to, saccharides, lipids, peptides, polypeptides and nucleic acids and derivatives thereof. Synthetic compound libraries are commercially available.

Alternatively, libraries of natural compounds in the form of bacterial, fungal, plant, and animal extracts are commercially available from a number of sources. In addition, natural and synthetically produced libraries can be generated according to methods known in the art, e.g., by standard extraction and fractionation methods. Furthermore any library or compound may readily be modified using standard chemical, physical, or biochemical methods.

Another class of agents that can be screened are antibodies, in particular monoclonal antibodies. These include certain antibodies targeting receptors that are overexpressed in cancer cells.

One of skill in the art will appreciate that the LSD1 inhibitors can be used alone or in combination with other compounds and therapeutic regimens to inhibit tumorigensis.

An effective amount of the inhibitor will be determined by the existence, nature, and extent of any adverse side-effects that accompany the administration of the composition; the LD₅₀ of the composition; and the side-effects of the composition at various concentrations. Typically, the amount of the composition administered will range from about 0.01 to about 20 mg per kg, more typically about 0.05 to about 15 mg per kg, even more typically about 0.1 to about 10 mg per kg body weight.

The inhibitor can be administered, for example, by intravenous infusion, orally, intraperitoneally, or subcutaneously. Oral administration is the preferred method of administration. The formulations of compounds can be presented in unit-dose or multi-dose sealed containers, such as ampoules and vials.

The LSD1 inhibitors are typically formulated with a pharmaceutically acceptable carrier before administration to an individual or subject. Pharmaceutically acceptable carriers are determined, in part, by the particular composition being administered, as well as by the particular method used to administer the composition. Accordingly, there are a wide variety of suitable formulations of pharmaceutical compositions of the present invention (see, e.g., Remington's Pharmaceutical Sciences, 17th ed., 1989).

### EXAMPLES

### Example 1

### Generation of the Targeting Vector

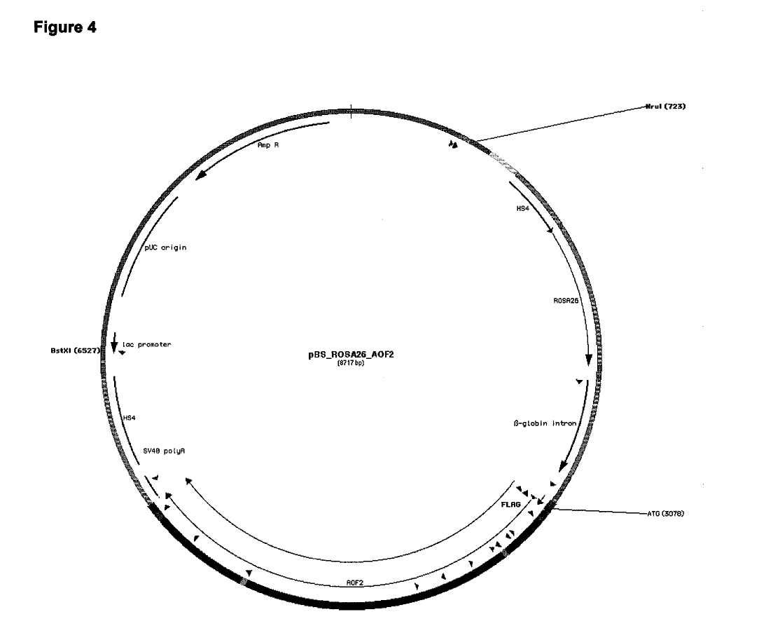

To generate the vector for ubiquitous expression of human Flag tagged LSD1 in transgenic mice the following cloning steps were made. Two copies of the core chicken β-globin insulator element HS4 from the Gary Felsenfeld laboratory (pNI-CD) were cloned into pSL301 using KpnI (pSL301-HS4). The insulator element was than isolated from this vector with EcoRV and cloned into a blunted NotI site in pSL301-TG-P containing an ARR₂Pb promoter, a rabbit β-globin intron and a SV40 poly A site (pSL301-HS4-TG-P). In parallel LSD1 was cloned from pCMX-Flag-Namo with EcoRV and blunted NheI into blunted BamHI and BglII sites from pBS-β-pA-HS4 containing two copies of the core chicken β-globin insulator element HS4 (pBS-Namo-HS4). The letter vector linearized with EcoRV and the insert from pSL301-HS4-TG-P digested with EcoRV and blunted XhoI were ligated to generate pBS-ARR₂Pb-AOF2. Than, pBS-ARR₂Pb-AOF2 digested with blunted XbaI to remove the ARR₂Pb promoter was ligated to the insert of pROSA26 promoter (Zambrowicz et al., 1997; Kisseberth et al., 1999) digested with blunted XbaI and SalI to generate pBS-ROSA26-AOF2 (SEQ ID NO:6). The map of the targeting vector is shown in FIG. 4. For microinjection the insert was relieved with MluI and BstXI.

### Example 2

### Generation of Transgenic Mice

All animals were housed in the experimental unit of the pathogen free barrier facility of the Central Clinical Research of the Freiburg University Medical Center in accordance with institutional guidelines and approved by the regional board. Transgenic mice were generated by pronuclear injection into fertilized eggs of FVB (Taketo et al. 1991) mice using standard procedures (Hogan et al. 1994). Three independent founder lines were analyzed. Genotyping and specificity of transgene expression was verified by PCR and RT-PCR, respectively in all 3 independent lines in RNA extracts of different organs. The following primers were used for genotyping generating a 153 bp fragment using standard PCR conditions: 5′-AATGCCTTCGAATTCAGCAC-3′ (SEQ ID NO 8); 5′-CCTTGTCATCGTCGTCCTTG-3′ (SEQ ID NO 9). The following primers were used for RT-PCR amplifying a 404 bp fragment of Flag-tagged LSD1 using standard conditions with 3% DMSO: 5′-gactacaaggacgacgat-3′ (SEQ ID NO 11); 5′-CCGCTCGAGTCAGCTTTCAT CCATCTCTCTG-3′ (SEQ ID NO 11).

### Example 3

### Detection Off LSD 1 Protein in Various Tissues of the Transgenic Mice

293 cells were transfected with 5 μg of pCMX-Flag-Namo. Protein extract was prepared 24 h after transfection using SC buffer containing protease inhibitors. Protein extracts from the indicated mouse tissues were dissolved in SC buffer containing protease inhibitors after homogenization in liquid nitrogen using a mortar and a pistil. Following preclearing with a 40 μl 1:1 slurry of GammaBind-Sepharose (Pharmacia), 2 mg of mouse tissue protein supernatants were incubated for 2.5 h with M2 α-Flag antibody (Sigma). Beads were washed five times with WB (10 mM Tris-HCl pH 8.0, 250 mM NaCl, 0.5% NP-40, 0.1 μg/μl bovine serum albumin, 0.5 mM Pefabloc) and analysed on a 10% SDS gel. Western blots were decorated with M2 antibody. Secondary antibody and chemiluminescence procedures were performed according to the manufacturer (Amersham).

Human FLAG-tagged LSD1 protein could be detected in various tissues from Rosa26-LSD1 transgenic mice, see FIG. 2.

### Example 4

### Detection of Tumors in the Transgenic Mice

Adult mice were killed by cervical dislocation. Various organs were dissected and photographed. Specimen were embed in paraffin after fixation in 4% PFA over night, washing in PBS, dehydration in Ethanol and xylene. Sections of 6 μm thickness were generated with a microtome. Sections were dehydrated and stained with eosin and hematoxylin using standard procedures.

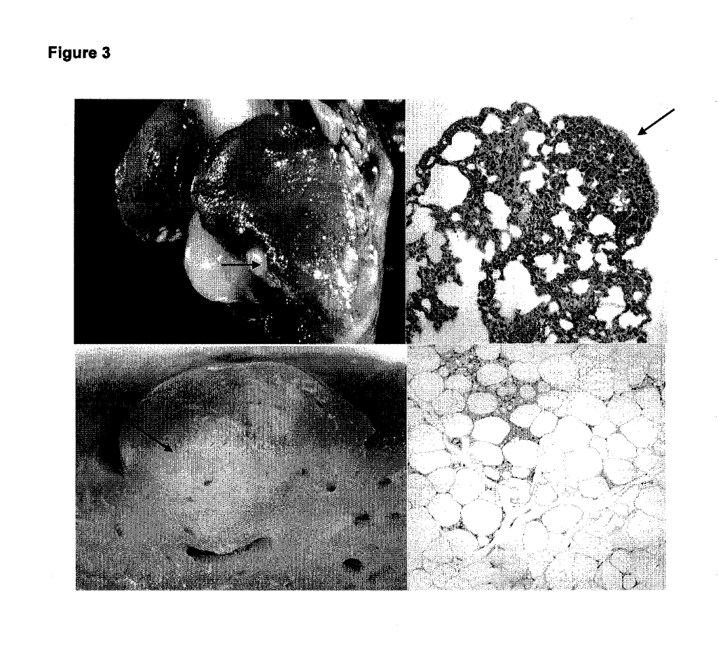

Analysis revealed the development of various tumors in the adult Rosa26-LSD1 transgenic mice, see FIG. 3. Macroscopic (upper left panel) and microscopic (upper right panel) photographs of a bronchial adenocarcinoma are shown (arrows). Macroscopic image of a section through a liver lobe (bottom left panel) with hepatocellular carcinoma (arrow) is shown. The bottom right panel depicts a section through a peritoneal lipoma stained by haematoxylin and eosin.

### REFERENCES

Chau, C. M., Deng, Z., Kang, H., Lieberman, P. M. (2008). Cell cycle association of the retinoblastoma protein Rb and the histone demethylase LSD1 with the Epstein-Barr virus latency promoter Cp. J. Virol. 82:3428-37.
Forneris, F., Binda, C., Battaglioli, E., Mattevi, A. (2008). LSD1: oxidative chemistry for multifaceted functions in chromatin regulation. Trends Biochem. Sci. 33:181-9.
Hogan, B., Constantini, F., Beddington, R. (1994). Manipulating the mouse embryo. Cold Spring Laboratory Press, Woodbury N.Y., 2^{nd} edition.
Huang, J., Sengupta, R., Espejo, A. B., Lee, M. G., Dorsey, J. A., Richter, M., Opravil, S., Shiekhattar, R., Bedford, M. T., Jenuwein, T., Berger, S. L. (2007). p53 is regulated by the lysine demethylase LSD1. Nature 449:105-8.
Kahl, P., Gullotti, L., Heukamp, L. C., Wolf, S., Friedrichs, N., Vorreuther, R., Solleder, G., Bastian, P. J., Ellinger, J., Metzger, E., Schüle R., Buettner R. (2006). Androgen receptor coactivators lysine-specific histone demethylase 1 and four and a half LIM domain protein 2 predict risk of prostate cancer recurrence. Cancer Res. 66:11341-7.
Kouzarides, T. (2007). Chromatin modifications and their function. Cell 128: 693-705.
Kisseberth, W. C., Brettingen, N. T., Lohse J. K., Sandgren, E. P. (1999). Ubiquitous expression of marker transgenes in mice and rats. Dev. Biol. 214:128-38.
Lee, M. G., Wynder, C., Cooch, N., Shiekhattar, R. (2005). An essential role for CoREST in nucleosomal histone 3 lysine 4 demethylation. Nature 437:432-5.
Metzger, E., Wissmann, M., Yin, N., Müller, J. M., Schneider, R., Peters, A. H., Günther, T., Buettner, R., Schüle, R. (2005). LSD1 demethylates repressive histone marks to promote androgen-receptor-dependent transcription. Nature 437:436-9.
Sharifi, N., Gulley, J. L., Dahut, W. L. (2005). Androgen deprivation therapy for prostate cancer. JAMA 294:238-44.
Shi, Y., Lan, F., Matson, C., Mulligan, P., Whetstine, J. R., Cole, P. A., Casero, R. A., Shi, Y. (2004). Histone demethylation mediated by the nuclear amine oxidase homolog LSD1. Cell 119:941-53.
Spannhoff, A., Heinke, R., Bauer, I., Trojer, P., Metzger, E., Gust, R., Schüle, R., Brosch, G., Sippl, W., Jung, M. (2007). Target-based approach to inhibitors of histone arginine methyltransferases. J. Med. Chem. 50:2319-25.
Taketo M., Schroeder A. C., Mobraaten L. E., Gunning K. B., Hanten G., Fox R. R., Roderick T. H., Stewart C. L., Lilly F., Hansen C. T., and Overbeek P. A. (1991) FVB/N: An inbred mouse strain preferable for transgenic analyses. Proc. Natl. Acad. Sci. USA 88, 2065-2069.
Wang, J., Scully, K., Zhu, X., Cai, L., Zhang, J., Prefontaine, G. G., Krones, A., Ohgi, K. A., Zhu, P., Garcia-Bassets, I., Liu, F., Taylor, H., Lozach, J., Jayes, F. L., Korach, K. S., Glass, C. K., Fu, X. D., Rosenfeld, M. G. (2007) Opposing LSD1 complexes function in developmental gene activation and repression programmes. Nature. 446:882-7.
Wissmann, M., Yin, N., Müller, J. M., Greschik, H., Fodor, B. D., Jenuwein, T., Vogler, C., Schneider, R., Günther, T., Buettner, R., Metzger E., Schüle R. (2007). Cooperative demethylation by JMJD2C and LSD1 promotes androgen receptor-dependent gene expression. Nat. Cell Biol. 9: 347-53.
Zambrowicz, B. P., Imamoto, A., Fiering, S., Herzenberg, L. A., Kerr, W. G., Soriano, P. (1997). Disruption of overlapping transcripts in the ROSA beta geo 26 gene trap strain leads to widespread expression of beta-galactosidase in mouse embryos and hematopoietic cells. Proc. Natl. Acad. Sci. USA. 94:3789-94.

1. A non-human transgenic animal whose genome comprises a stably integrated transgenic nucleotide sequence encoding Lysine-specific Demethylase 1 (LSD1) operably linked to a promoter.

2. The transgenic animal of claim 1, wherein said LSD1 is human LSD1.

3. The transgenic animal of claim 1, wherein said LSD1 comprises at least amino acids 2 to 852 of the amino acid sequence set forth in SEQ ID NO: 3.

4. The transgenic animal according to claim 1, wherein said transgenic nucleotide sequence comprises nucleotides 4 to 2556 of the nucleotide sequence set forth in SEQ ID NO: 1.

5. The transgenic animal according to claim 1, wherein said LSD1 is overexpressed in the transgenic animal.

6. The transgenic animal of claim 5, wherein said overexpression is present at least in skeletal muscle, prostate tissue, testis and heart muscle.

7. The transgenic animal according to claim 1, wherein said promoter is a Rosa26 promoter.

8. The transgenic animal according to claim 1, wherein said animal develops at least one tumor during its life.

9. The transgenic animal according to claim 8, wherein said tumor is selected from the group consisting of lung tumors, hepatocellular carcinoma and peritoneal lipoma.

10. The transgenic animal according to claim 1, wherein said transgenic animal is a rodent.

11. The transgenic animal according to claim 1, wherein said animal does not comprise other transgenic nucleotide sequences.

12. A method for generating a non-human transgenic animal according to claim 1, said method comprising the step of: transfecting a target animal with a vector molecule comprising an expression cassette, wherein said expression cassette comprises a polynucleotide encoding LSD 1 operably linked to a promoter.

13. A method for identifying a compound that inhibits tumor growth, said method comprising the steps of: (a) administering a test compound to a transgenic animal according to claim 1 and (b) determining the effect of the test compound on the initiation, maintenance, or progression of cancer in said transgenic animal, thereby identifying a compound that inhibits tumor growth.

14. A method according to claim 13, wherein said test compound is selected from the group consisting of LSD1 inhibitors, modulators of an androgen receptor, modulators of p53, and modulators of Rb.

15. The non-human transgenic animal according to claim 1, wherein said transgenic animal is an animal model for cancer.

16. The transgenic animal according to claim 1, wherein said transgenic animal is a mouse.


SEQUENCE LISTING (11 sequences)

SEQ ID NO: 1 - DNA, 2559 bp, homo sapiens
atgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcgggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtga

SEQ ID NO: 2 - DNA, 3053 bp, homo sapiens
  CDS (150)..(2708)
ggcgcggcgggagcgcgcttggcgcgtgcgtacgcgacggcggttggcggcgcgcgggcagcgtgaagcgaggcgaggcaaggcttttcggacccacggagcgacagagcgagcggcccctacggccgtcggcggcccggcggcccgagatgttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcgggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtgagacagatgcattctaagggaagaggcccatgtgcctgtttctgccatgtaaggaaggctcttctagcaatactagatcccactgagaaaatccaccctggcatctgggctcctgatcagctgatggagctcctgatttgacaaaggagcttgcctcctttgaatgacctagagcacagggaggaacttgtccattagtttggaattgtgttcttcgtaaagactgaggcaagcaagtgctgtgaaataacatcatcttagtcccttggtgtgtggggtttttgttttttttttatattttgagaataaaacttcatataaaattggcaaaaaaaaaaaaaaaaaa

SEQ ID NO: 3 - protein, 852 aa, homo sapiens
MLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 4 - DNA, 3030 bp, mus musculus
  CDS (139)..(2700)
gggcgcgtgcgcacgcgggggtgtttggcttcgcacggagcgtgagaggtgcggggcggagaggcgcgaggcggctgcggacccacggagcggcagaccgatcggcccctgcggcccgcggcggccaggcggcccgagatgttgtctgggaagaaggcggcggcggcggcagcggcagcggcggcggcggcggctgctgggaccgaggccgggtccggggcggcgggcggtgccgagaacggctctgaggtggccgcgccgcccgcgggcctgacgggccccaccgacatggctacgggggcggcgggcgagcgcactccccgaaagaaggagcctccgcgggcctcgccgcccgggggcctagccgagccgccggggtctgctgggccccaggcggggcccacagccgggcccggctccgcgacgcccatggagaccggaatagccgagaccccggagggccgacggaccagccggcgcaagcgggccaaggtagaatacagagaaatggatgaaagcttggccaacctctcagaagatgaatattattcggaagaagaaagaaatgctaaagcagagaaggaaaagaagcttcccccaccacctcctcaagccccacctgaggaagaaaatgaaagtgagccggaagagccgtctggtgtggagggtgcagcttttcaaagccgacttccccatgaccgaatgacctctcaggaagcagcctgtttcccagacatcatcagtgggcctcagcagacacagaaggtttttctgttcatcaggaatcgcacattgcagttatggctggacaacccaaagatccagctgacgtttgaagccactctccagcagctggaagcgccttacaacagcgatactgtgcttgtccaccgagttcacagttacttagagcgccatggtcttatcaacttcggcatctacaagaggataaaacccttaccaattaaaaagacaggaaaggtgattattataggttcaggtgtttctggcttggcagcagctcgacagctacagagttttgggatggatgtcacacttctggaagccagggatcgagtaggtggacgagttgctacatttcgaaaaggaaactatgtagctgatcttggcgccatggttgtaacaggtcttggagggaatcccatggctgtcgtcagcaaacaagtaaatatggaactggccaagatcaagcaaaaatgcccactttatgaagccaatggacaagctgttccaaaagaaaaagatgaaatggtagaacaagaatttaaccggttgctagaagccacttcttaccttagtcaccagttagacttcaacgtcctcaataataaacctgtatcccttggccaggcattggaggttgtcattcagctgcaagaaaagcatgtcaaagatgagcagattgaacattggaagaagatagtgaaaactcaggaggagttgaaagagcttcttaataagatggtaaatttgaaggagaaaattaaagagctccatcagcaatacaaagaagcttcagaagtgaagccgcccagagatatcacagccgagttcctggtgaagagcaagcacagggacctgactgccctctgcaaggaatatgatgaattagctgaaacacaaggaaagctagaagaaaaacttcaagaattggaagccaatcccccaagtgatgtatacctctcatcaagagacagacaaatacttgactggcattttgcaaatcttgaatttgccaacgccacacctctctctaccctctctcttaaacattgggatcaggatgatgactttgagtttactggaagccacctgacagtaaggaatggctactcatgtgtgcctgtggctttagctgaaggcttggacattaaactgaacacagcagtgcggcaggttcgctacacagcctcaggatgtgaagtgattgctgtgaacacacgttccacaagtcaaacctttatttataagtgtgatgcagttctctgtacacttcctttgggagtgttgaagcagcagccaccagctgttcagtttgtgccacctcttcctgagtggaaaacatctgcagtccaaaggatgggatttggcaaccttaacaaggtggtgttatgctttgaccgtgtgttctgggacccaagtgtcaatttgtttgggcacgttggcagtacaactgctagcaggggtgagctcttcctcttctggaacctatataaagctccaatactattggccctggtagcaggagaagctgctggcattatggagaacattagtgatgatgtgattgtcggccggtgcctggccattctcaaagggatttttggcagcagtgcagtcccacagcccaaggaaactgtggtatctcgttggcgtgctgatccgtgggcccggggctcctattcttatgtggctgcaggatcctctggaaatgactatgatttaatggctcagccgatcactcctggcccctcaattccaggtgccccacagccaatcccaagactcttctttgctggagaacacacaatccggaactacccagctacagtccatggtgctctgttgagtgggcttcgagaagcaggaaggattgccgaccagtttttgggagccatgtacactttgcctcgtcaggccacaccaggtgtccctgcacagcagtccccaagtatgtgagacagatggttctgaacagagagatccaacggcatgtcatctgccacgtaagcaagctcttctagcaatactagatcctactgagaaactccatgtcatcagctactgggactcctagtttgacagcagaggctggctcctttggctgacagcaacttacccattgatttggaagtacagctccataaagactgctcgagaagcaagtggtgtgagataacctcttagtctatggtgtttgtttgtttttgtttttttttaatatattttgagaataaaactttaaaataattttatatgaaaatttatttttaaaaaaaaaaaaaaaaa

SEQ ID NO: 5 - protein, 853 aa, mus musculus
MLSGKKAAAAAAAAAAAAAAGTEAGSGAAGGAENGSEVAAPPAGLTGPTDMATGAAGERTPRKKEPPRASPPGGLAEPPGSAGPQAGPTAGPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPIKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 6 - DNA, 8717 bp, artificial sequence
  pBS-ROSA26-AOF2
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgattcgagctctgtacatgtccgcggtcgcgacgtacgcgtatcgatggcgccagctgcaggcggccgccatatgcatcctaggcctattaatattccggagtatacgtagccggctaacgttaacaaccggtaccgagttggcgcgcctgggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacgggcgcgcctgggagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctgggcgcgccaattaaccctcactaaagggggtacctctagtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgaggcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatccactagttctagaactatagctagcatgcgcaaatttaaagcgctgatatcgatcgcgcgcagatcctaagaacttccaggggaggtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacaccctggtcatcatcctgcctttctctttatggttacaatgatatacactgtttgagatgaggataaaatactctgagtccaaaccgggcccctctgctaaccatgttcatgccttcttctctttcctacagctcctgggcaacgtgctggttatgtgctgtctcatcaaatggcaaagaattcatggctggtgaccacgtcgtggaatgccttcgaattcagcacctgcacatgggacgtcgacctgaggtaattataacccgggccctatatatggatccagatcgatcatcaggatcggtaccgggccccccctcgagaagcttccaccatggactacaaggacgacgatgacaaggaattcttatctgggaagaaggcggcagccgcggcggcggcggctgcagcggcagcaaccgggacggaggctggccctgggacagcaggcggctccgagaacgggtctgaggtggccgcgcagcccgcgggcctgtcgggcccagccgaggtcgggccgggggcggtgggggagcgcacaccccgcaagaaagagcctccgcgggcctcgccccccgggggcctggcggaaccgccggggtccgcagggcctcaggccggccctactgtcgtgcctgggtctgcgacccccatggaaactggaatagcagagactccggaggggcgtcggaccagccggcgcaagcgggcgaaggtagagtacagagagatggatgaaagcttggccaacctctcagaagatgagtattattcagaagaagagagaaatgccaaagcagagaaggaaaagaagcttcccccaccaccccctcaagccccacctgaggaagaaaatgaaagtgagcctgaagaaccatcgggtgtggagggcgcagctttccagagccgacttcctcatgaccggatgacttctcaagaagcagcctgttttccagatattatcagtggaccacaacagacccagaaggtttttcttttcattagaaaccgcacactgcagttgtggttggataatccaaagattcagctgacatttgaggctactctccaacaattagaagcaccttataacagtgatactgtgcttgtccaccgagttcacagttatttagagcgtcatggtcttatcaacttcggcatctataagaggataaaacccctaccaactaaaaagacaggaaaggtaattattataggctctggggtctcaggcttggcagcagctcgacagttacaaagttttggaatggatgtcacacttttggaagccagggatcgtgtgggtggacgagttgccacatttcgcaaaggaaactatgtagctgatcttggagccatggtggtaacaggtcttggagggaatcctatggctgtggtcagcaaacaagtaaatatggaactggccaagatcaagcaaaaatgcccactttatgaagccaacggacaagctgttcctaaagagaaagatgaaatggtagagcaagagtttaaccggttgctagaagctacatcttaccttagtcatcaactagacttcaatgtcctcaataataagcctgtgtcccttggccaggcattggaagttgtcattcagttacaagagaagcatgtcaaagatgagcagattgaacattggaagaagatagtgaaaactcaggaagaattgaaagaacttcttaataagatggtaaatttgaaagagaaaattaaagaactccatcagcaatacaaagaagcatctgaagtaaagccacccagagatattactgccgagttcttagtgaaaagcaaacacagggatctgaccgccctatgcaaggaatatgatgaattagctgaaacacaaggaaagctagaagaaaaacttcaggagttggaagcgaatcccccaagtgatgtatatctctcatcaagagacagacaaatacttgattggcattttgcaaatcttgaatttgctaatgccacacctctctcaactctctcccttaagcactgggatcaggatgatgactttgagttcactggcagccacctgacagtaaggaatggctactcgtgtgtgcctgtggctttagcagaaggcctagacattaaactgaatacagcagtgcgacaggttcgctacacggcttcaggatgtgaagtgatagctgtgaatacccgctccacgagtcaaacctttatttataaatgcgacgcagttctctgtacccttcccctgggtgtgctgaagcagcagccaccagccgttcagtttgtgccacctctccctgagtggaaaacatctgcagtccaaaggatgggatttggcaaccttaacaaggtggtgttgtgttttgatcgggtgttctgggatccaagtgtcaatttgttcgggcatgttggcagtacgactgccagcaggggtgagctcttcctcttctggaacctctataaagctccaatactgttggcactagtggcaggagaagctgctggtatcatggaaaacataagtgacgatgtgattgttggccgatgcctggccattctcaaagggatttttggtagcagtgcagtacctcagcccaaagaaactgtggtgtctcgttggcgtgctgatccctgggctcggggctcttattcctatgttgctgcaggatcatctggaaatgactatgatttaatggctcagccaatcactcctggcccctcgattccaggtgccccacagccgattccacgactcttctttgcgggagaacatacgatccgtaactacccagccacagtgcatggtgctctgctgagtgggctgcgagaagcgggaagaattgcagaccagtttttgggggccatgtatacgctgcctcgccaggccacaccaggtgttcctgcacagcagtccccaagcatgtgagctaggatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgactctagcagtgaaagtctgcaatgaattcgagttggcgcgcctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctggtgggctttgggaggggggctgtccccgtgagctcccaggcgcgcctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctcccaggcgcgccaactcgctagaggtaccggttgttaacgttagccggctacgtatactccggaatattaataggcctaggatgcatatggcggccgccaccgcggtggagctccagcttttgttgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 7 - protein, 862 aa, artificial sequence
  Synthetic Construct
MDYKDDDDKEFLSGKKAAAAAAAAAAAATGTEAGPGTAGGSENGSEVAAQPAGLSGPAEVGPGAVGERTPRKKEPPRASPPGGLAEPPGSAGPQAGPTVVPGSATPMETGIAETPEGRRTSRRKRAKVEYREMDESLANLSEDEYYSEEERNAKAEKEKKLPPPPPQAPPEEENESEPEEPSGVEGAAFQSRLPHDRMTSQEAACFPDIISGPQQTQKVFLFIRNRTLQLWLDNPKIQLTFEATLQQLEAPYNSDTVLVHRVHSYLERHGLINFGIYKRIKPLPTKKTGKVIIIGSGVSGLAAARQLQSFGMDVTLLEARDRVGGRVATFRKGNYVADLGAMVVTGLGGNPMAVVSKQVNMELAKIKQKCPLYEANGQAVPKEKDEMVEQEFNRLLEATSYLSHQLDFNVLNNKPVSLGQALEVVIQLQEKHVKDEQIEHWKKIVKTQEELKELLNKMVNLKEKIKELHQQYKEASEVKPPRDITAEFLVKSKHRDLTALCKEYDELAETQGKLEEKLQELEANPPSDVYLSSRDRQILDWHFANLEFANATPLSTLSLKHWDQDDDFEFTGSHLTVRNGYSCVPVALAEGLDIKLNTAVRQVRYTASGCEVIAVNTRSTSQTFIYKCDAVLCTLPLGVLKQQPPAVQFVPPLPEWKTSAVQRMGFGNLNKVVLCFDRVFWDPSVNLFGHVGSTTASRGELFLFWNLYKAPILLALVAGEAAGIMENISDDVIVGRCLAILKGIFGSSAVPQPKETVVSRWRADPWARGSYSYVAAGSSGNDYDLMAQPITPGPSIPGAPQPIPRLFFAGEHTIRNYPATVHGALLSGLREAGRIADQFLGAMYTLPRQATPGVPAQQSPSM

SEQ ID NO: 8 - DNA, 20 bp, artificial sequence
  primer 1
aatgccttcgaattcagcac

SEQ ID NO: 9 - DNA, 20 bp, artificial sequence
  primer 2
ccttgtcatcgtcgtccttg

SEQ ID NO: 10 - DNA, 18 bp, artificial sequence
  primer 3
gactacaaggacgacgat

SEQ ID NO: 11 - DNA, 31 bp, artificial sequence
  primer 4
ccgctcgagtcagctttcatccatctctctg